(19) United States
(12) Patent Application Publication
(10) Pub. No.: US 2009/0105708 A1
(43) Pub. Date: Apr. 23, 2009

(54) DUAL FREQUENCY LED/ELECTRODE SURGICAL DEVICE, KIT AND METHOD

(76) Inventors: William J. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/876,897

(22) Filed: Oct. 23, 2007

(51) Int. Cl. A61B 18/14; A61B 18/18

(52) U.S. Cl. 606/34; 606/10

(57) ABSTRACT

A surgical tool device, a kit and a method are described which provide a novel dual frequency LED/electrode scheme for use in manipulating nerve and innervated structures. The surgical tool device includes a probe assembly coupled to a handle assembly. The probe assembly has a low frequency light emitting diode (LED), a high frequency LED, and a stimulator electrode. The low frequency LED is used to promote healing and the high frequency LED is to aid in promoting a microbe free surgical area. The handle assembly has a system on a chip (SOC) electrically coupled to the low frequency LED, to the high frequency LED, and to the stimulator electrode. The kit includes the unattached components of the device and may also include an detector electrode probe along with an optional monitoring system. The method includes the steps of adjoining, affixing, attaching, and obtaining.

### FIELD OF THE INVENTION

The present invention relates to a surgical tool device, kit and method thereof. More particularly, the present invention is directed at a surgical tool device having two different frequency light sources and having a stimulating electrode, a kit and a method thereof for use in performing surgical procedures at or near nerve structures.

### BACKGROUND OF THE INVENTION

Nearly every surgical operation involves establishing some sort of opening or channel to gain access into and around a given surgical target site. Oftentimes, based on the anatomical location of the surgical target site, surgical tools are required to maintain this working channel within a close proximity to various nerve structures and bundles. Accordingly, the surgeon must be diligent in avoiding, or at least in minimizing, any contact with these exposed nerve structures to avoid injuring the exposed nerve structures.

Therefore, a need exists for a new and improved dual frequency LED/electrode surgical device having a low frequency LED, a high frequency LED and a stimulator electrode. In this respect, the dual frequency LED/electrode surgical device according to the present invention substantially departs from the conventional concepts and designs of the prior art, and in doing so provides an apparatus primarily developed for the purpose of providing a convenient means for making it possible to promoting healing and sanitizing a surgical wound as well as providing a means for identifying a particular nerve structure.

### SUMMARY OF THE INVENTION

The present device, kit and method of using, according to the principles of the present invention, overcomes a number of the shortcomings of the prior art by providing a novel dual frequency LED/electrode surgical device, kit and method for use in promoting healing and sanitizing a surgical wound as well as providing a means for identifying a particular nerve structure. The device includes a probe having a low frequency LED, a high frequency LED and a stimulator electrode. The kit includes the un-interconnected elements of the device. The method includes the steps of adjoining, affixing, attaching, and obtaining.

The present invention provides an improved dual frequency LED/electrode surgical device, which will be described subsequently in great detail, that provides a new and improved dual frequency LED/electrode surgical device which is not anticipated, rendered obvious, suggested, or even implied by the prior art, either alone or in any combination thereof.

The present invention essentially comprises a dual frequency LED/electrode surgical device that includes a probe assembly coupled to a handle assembly. The probe assembly has a low frequency light emitting diode (LED), a high frequency LED, and a stimulator electrode. The low frequency LED is used to thought to promote healing and the high frequency LED is thought to promote a microbe free surgical area. The handle assembly has a system on a chip (SOC) electrically coupled to the low frequency LED, to the high frequency LED, and to the stimulator electrode. The kit includes the unattached components of the device and may also include an detector electrode probe along with an optional monitoring system. The method includes the steps of adjoining, affixing, attaching, and obtaining.

There has thus been outlined, rather broadly, the more important features of the invention in order that the detailed description thereof that follows may be better understood, and in order that the present contribution of the art may be better appreciated.

The invention of the device may also optionally include a plurality of control switches and a power supply. The invention of the kit may also optionally include a detector electrode probe, a cable, and a monitoring system. The invention of the method may also optionally include the steps of aligning, dispensing, displaying, emitting, irradiating, inserting, making, pressing, pulling, stimulating, and withdrawing.

The present invention provides a dual frequency LED/electrode surgical device that provides in the apparatuses and methods of the prior art some of the advantages thererof, while simultaneously overcoming some of the disadvantages normally associated therewith.

Also the present invention provides a kit comprising the non connected components of the dual frequency LED/electrode surgical device.

Lastly, the present invention provides a new and improved method of using comprising the steps of adjoining, affixing, attaching, and obtaining.

Numerous other features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These and other features of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood will become apparent when consideration is given to the following detailed description thereof. Such description makes reference to the annexed drawings wherein:

FIG. 1 is a perspective view of an embodiment of the surgical tool device constructed in accordance with the principles of the present invention;

FIGS. 2A, 2B, and 2C depict various probe assembly designs of the surgical tool device of the present invention;

FIGS. 3A, 3B, and 3C depict a close up partial view of some of the various engagement surfaces of the probe assembly 12 of the surgical tool device 10;

FIG. 4 depicts a perspective view of an assembled kit of the surgical tool device;

FIG. 5 depicts a logical communication scheme of the surgical tool device;

FIG. 6 depicts a logical communication scheme of the kit for the surgical tool device; and

FIGS. 7A, 7B, 7C, 7D, 7E, 7F, 7G, 7H, and 7I depict various electronic schemes for driving the low and high frequency LEDS of the surgical tool device.

The same reference numerals refer to the same parts throughout the various figures.

### DETAILED DESCRIPTION

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

Accordingly, the detailed discussion herein of one or more embodiments is not intended, nor is to be construed, to limit the metes and bounds of the patent protection afforded the present invention, in which the scope of patent protection is intended to be defined by the claims and their equivalents thereof. Therefore, embodiments not specifically addressed herein, such as adaptations, variations, modifications, and equivalent arrangements, should be and are considered to be implicitly disclosed by the illustrative embodiments and claims described herein and therefore fall within the scope of the present invention.

Further, it should be understood that, although steps of various the claimed method may be shown and described as being in a sequence or temporal order, the steps of any such method are not limited to being carried out in any particular sequence or order, absent an indication otherwise. That is, the claimed method steps are to be considered to be capable of being carried out in any sequential combination or permutation order while still falling within the scope of the present invention.

Additionally, it is important to note that each term used herein refers to that which a person skilled in the relevant art would understand such term to mean based on the contextual use of such term herein. To the extent that the meaning of a term used herein, as understood by the person skilled in the relevant art based on the contextual use of such term, differs in any way from any particular dictionary definition of such term, it is intended that the meaning of the term as understood by the person skilled in the relevant art should prevail.

Furthermore, a person skilled in the art of reading claimed inventions should understand that “a” and “an” each generally denotes “at least one,” but does not exclude a plurality unless the contextual use dictates otherwise. And that the term “or” denotes “at least one of the items,” but does not exclude a plurality of items of the list.

Unless otherwise defined, all scientific and technical terms used herein are to be construed as having the same meaning as commonly understood by one of ordinary skill in the art to which this invention pertains. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present document, including definitions, will control. Unless otherwise indicated, materials, methods, and examples described herein are illustrative only and not intended to be limiting.

It will be understood that light will be defined herein as an electromagnetic radiation ranging from infrared, visible to ultraviolet wavelengths. Accordingly, the term light will be defined as any electromagnetic radiation ranging between about 250 nm to 1,500 nm.

It will be understood that a photosensitizer will be defined herein as a chemical compound that produces a biological effect upon photoactivation, or a biological precursor of a compound that produces a biological effect upon photoactivation. The photosensitizer must have a sufficiently low toxicity to permit administration of the photosensitizer to the patient within an acceptable level of safety. Preferably, the photosensitizer is essentially nontoxic, however due to their polycyclic aromatic nature or their multiple conjugated rings that allow for light absorption, fluorescence, phosphorescence and photoactivation, some of these photosensitizers may exhibit some toxicity.

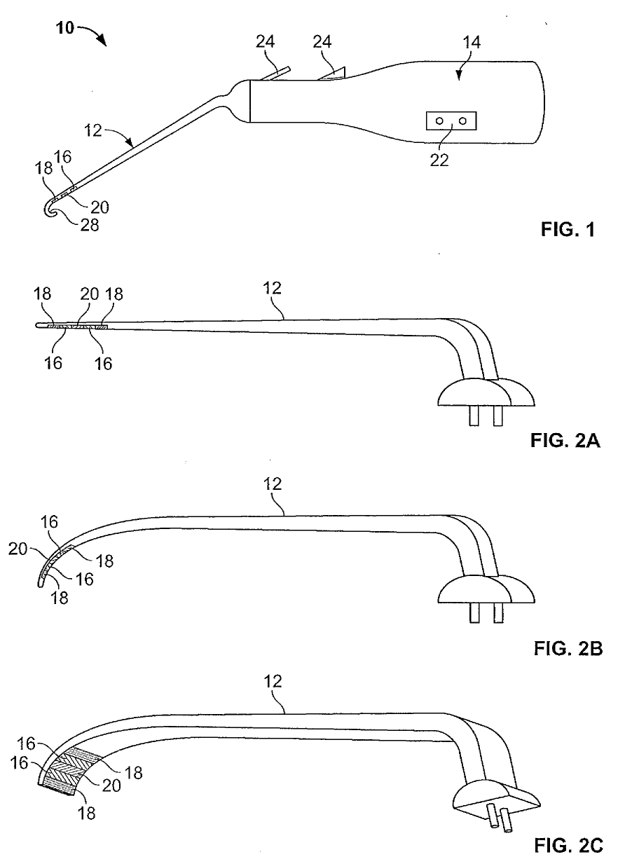

Referring now to the drawings, and in particular FIGS. 1 to 7 thereof, one preferred embodiment of the present invention is shown and generally designated by the reference numeral 10. The same reference numerals refer to the same parts throughout the various figures.

One preferred embodiment of a surgical tool device 10 comprises a probe assembly 12 coupled to a handle assembly 14, in which the probe assembly 12 has a low frequency light emitting diode (LED) 16, a high frequency LED 18, and a stimulator electrode 20; and the handle assembly 14 has a system on a chip (SOC) 22 electrically coupled to the low frequency LED 16, to the high frequency LED 18, and to the stimulator electrode 20.

The low frequency LED 16 may be any commercially available low frequency LED 16. One embodiment is that the low frequency LED 16 is configured to emit light between red and infrared. One embodiment is that the low frequency LED 16 may be configured to emit monochromatic light within a wavelength range between about 600 nm to about 1000 nm. Another embodiment is that the low frequency LED 16 may be configured to emit a spectral band of light of at least 10 nm wide within a wavelength range between about 600 nm to about 1000 nm. Still another embodiment is that the low frequency LED 16 may be configured to emit light at about 1 μW/cm² to about 1 W/cm².

The high frequency LED 16 may be any commercially available high frequency LED 16. One embodiment of the high frequency LED 18 is that it is configured to emit light between green and ultraviolet. A more preferred embodiment of the high frequency LED 18 is that it is configured to emit monochromatic light within a wavelength range between about 250 nm to about 550 nm. Another embodiment of the high frequency LED 18 is that it is configured to emit a spectral band of light of at least 10 nm wide within a wavelength range between about 250 nm to about 550 nm. Still another embodiment is that the high frequency LED 18 is that it is configured to emit light at about 1 μW/cm² to about 1 W/cm².

The probe assembly 12 of the surgical tool device 10 may be shaped and designed in any configuration suitable for surgical purposes. One preferred embodiment of the shape of the probe assembly is that it has an arcuate engagement surface 28.

The stimulator electrode 20 may be powered by AC or DC current or voltage signals driven and coordinated by the SOC.

Another embodiment of the surgical tool device 10 comprises the probe assembly 12 having the low frequency LED 16, the high frequency LED 18, the stimulator electrode 20 and the SOC 22 attached to the probe assembly 12. The SOC 22 is electrically coupled to the low frequency LED 16, to the high frequency LED 18, and to the stimulator electrode 20. Finally, the handle assembly 14 is attached to the probe assembly 12.

An optional plurality of control switches 24 may be added to the surgical tool device 10. One embodiment of the optional control switches is that it is attached to the handle so that the control switches 24 are electrically coupled to the SOC 22, to the low frequency LED 16, to the high frequency LED 18, and to the stimulator electrode 20.

An optional power supply 26 may be added to the surgical tool device 10. One embodiment is that the optional power supply 26 is attached to the handle so that the power supply 26 is electrically coupled to the SOC 22, to the low frequency LED 16, to the high frequency LED 18, and to the stimulator electrode 20. The power supply is selected from the group consisting of a battery power supply and a high capacity capacitor power supply.

One preferred embodiment of the kit for the surgical tool device 10 comprises the unattached probe assembly 12 and the handle assembly 14 of the surgical tool device 10. The probe assembly 12 of the kit has the low frequency LED 16, the high frequency LED 18, and the stimulator electrode 20 attached to the probe assembly 12. The handle assembly 14 is configured to be attached to the probe assembly 12, in which the handle assembly 14 has an SOC 22, a a plurality of control switches 24, and a power supply 26. The SOC 22 is configured to be electrically coupled to the low frequency LED 16, to the high frequency LED 18, and to the stimulator electrode 20. The control switches 24 are attached to the handle, in which the control switches 24 are configured to be electrically coupled to the SOC 22, to the low frequency LED 16, to the high frequency LED 18, and to the stimulator electrode 20. The power supply 26 is attached to the handle, in which the power supply 26 electrically coupled to the SOC 22, to the low frequency LED 16, to the high frequency LED 18, and to the stimulator electrode 20.

An optional detector electrode probe 30 may be added to the kit for the surgical tool device 10 in which the detector electrode probe 30 is configured to be electrically coupled to the SOC 22.

An optional cable 32 may be added to the kit for the surgical tool device 10 in which the cable 32 is configured to electrically couple together the detector electrode probe 30 to the SOC 22, and to the stimulator electrode 20.

An optional monitoring system 34 may be added to the kit for the surgical tool device 10 in which the monitoring system 34 is configured to be electrically coupled to the detector electrode.

One preferred embodiment of a method of using a kit for the surgical tool device 10 comprising the steps of adjoining, affixing, attaching, and obtaining. The step comprises obtaining the kit comprising: a probe assembly 12 having: a low frequency light emitting diode (LED) 16 attached to the probe assembly 12; a high frequency LED 18 attached to the probe assembly 12; and a stimulator electrode 20 attached to the probe assembly 12; a handle assembly 14 configured to be attached to the probe assembly 12, the handle assembly 14 having: a system on a chip attached to the handle, the SOC 22 configured to be electrically coupled to the low frequency LED 16, to the high frequency LED 18, and to the stimulator electrode 20; a plurality of control switches 24 attached to the handle, the control switches 24 electrically coupled to the SOC 22; and a power supply 26 attached to the handle, the power supply 26 electrically coupled to the SOC 22, and to the control switches 24; a detector electrode probe 30 configured to be electrically coupled to the SOC 22; a cable 32 configured to electrically couple together the detector electrode probe 30 to the SOC 22, to the stimulator electrode 20; and a monitoring system 34 configured to be electrically coupled to the detector electrode. The attaching step comprises attaching operatively together the probe assembly 12 to the handle assembly 14 so that the SOC 22 is electrically coupled to the low frequency LED 16, to the high frequency LED 18, to the stimulator electrode 20, to the power supply 26, and to the control switches 24. The adjoining step comprises adjoining operatively together the cable 32 to the handle and to the detector electrode so that the SOC 22 is electrically coupled to the detector electrode. The affixing step comprises affixing together the detector electrode to the monitoring system 34 so that the detector electrode and the stimulator electrode 20 are operatively coupled to the monitoring system 34.

The medical procedure supported by this method may be anyone of the following surgical procedures and non-surgical procedures, such as, endoscopic procedures, fluoroscopic procedures, stent delivery procedures, aortic aneurysm repairs, cranial aneurysm repairs, delivery of drugs, delivery of biological agents, cardiac surgery with cardiopulmonary bypass circuits, cardiac surgery without cardiopulmonary bypass circuits, brain surgery, cardiograms, heart valve repair, heart valve replacement, revascularization procedures, transmyocardial revascularization, percutaneous myocardial revascularization, anstomosis procedures, beating heart surgery, vascular surgery, neurosurgery, electrophysiology procedures, dianostic procedures, therapeutic procedures, ablation procedures, ablation of arrhythmias, endovascular procedures, treatment of the liver, treatment of the spleen, treatment of the heart, treatment of the lungs, treatment of major blood vessels, noninvasive procedures, invasive procedures, imaging procedures, CAT scan procedures, MRI procedures, gene therapy procedures, cellular therapy procedures, cancer therapy procedures, radiation therapy procedures, transplantation procedures, coronary angioplast procedures, atherectomy procedures, atherosclerotic place removal procedures, birthing procedures, spinal cord procedures including intrathecal access, epidural access and transcutaneous access.

An optional set of steps may be added to the method to further comprise the steps of aligning, dispensing, displaying, emitting, irradiating, inserting, making, pressing, pulling, stimulating, and withdrawing. The making step comprises making a opening into flesh near a nerve. The dispensing step comprises dispensing an aliquot of a photosensitizer into the opening. The inserting step comprises inserting the probe assembly 12 into an opening. The aligning step comprises aligning the probe adjacent to the nerve while the probe is inserted into the opening. The pressing step comprises pressing on one control switch 24 to activate the stimulator electrodes 20 to produce electrical impulses while the probe is aligned adjacent to the nerve. The stimulating step comprises stimulating the nerve with the electrical impulses. The displaying step comprises displaying a response of the detector electrode while stimulating the nerve to identify the nerve and to verify the alignment of the probe. The irradiating step comprises irradiating low frequency light from the low frequency LED 16 onto the nerve while the probe is aligned next to the nerve. The emitting step comprises emitting high frequency light from the high frequency LED 18 onto the nerve while the probe is aligned next to the nerve. The pulling step comprises pulling the nerve aside with the probe assembly 12. The withdrawing step comprises withdrawing the probe assembly 12 from the opening.

Any number of photosensitizers can be used in conjunction in practicing with the claimed method of the present invention. They differ in the properties of light absorption and fluorescence, biodistribution, temporal uptake, clearance, and mechanisms of photoactivatable cytotoxicity. Classes of photosensitizers include acridine dyes, bacteriochlorins, bacteriochlorophylls, chlorins, hematoporphyrins, phthalocyanines, porphyrins, purpurins, naphthalocyanines, non-tetrapyrrole photosensitizers, texaphyrins, uroporphyrins.

Referring now to FIG. 1 that depicts a perspective view of an embodiment of the surgical tool device showing the surgical tool device 10 having a probe assembly 12 coupled to a handle assembly 14. The probe assembly 12 is shown having a low frequency LED 16, a high frequency LED 18, and a stimulator electrode 20. The handle assembly 14 is shown having a system on a chip (SOC) 22 and having plurality of control switches 24. The SOC 22 of the handle assembly is electrically coupled to the low frequency LED 16, to the high frequency LED 18, to the stimulator electrode 20 and to the control switches 24.

Referring now to FIG. 2A, 2B, and 2C that depict various probe assembly 12 designs of the surgical tool device 10. The probe assembly 12 is shown having any number of designs, including a curved nerve hook design as in FIGS. 2B and 2C as well as a straight needle design as shown in FIG. 2A. Also shown in FIGS. 2A-2C are the low frequency LED 16, the high frequency LED 18, and a stimulator electrode 20 attached to the probe assembly 12.

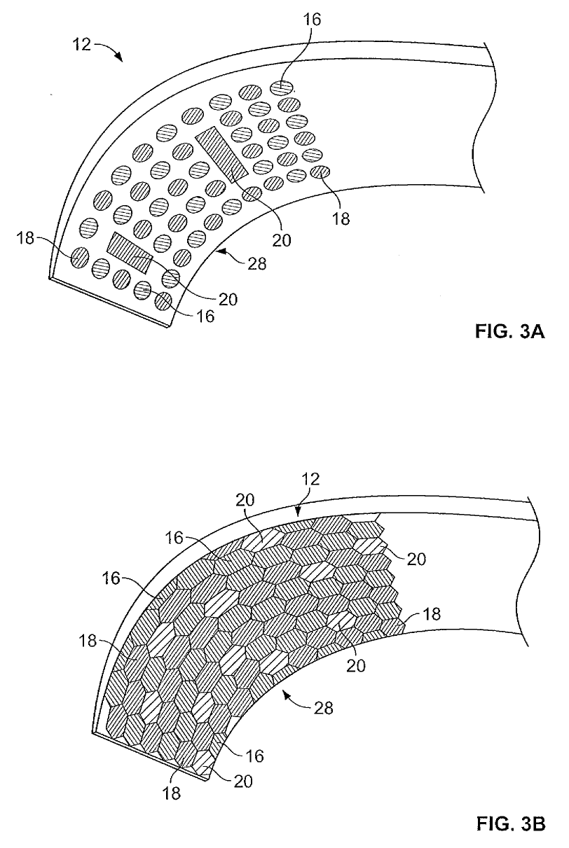

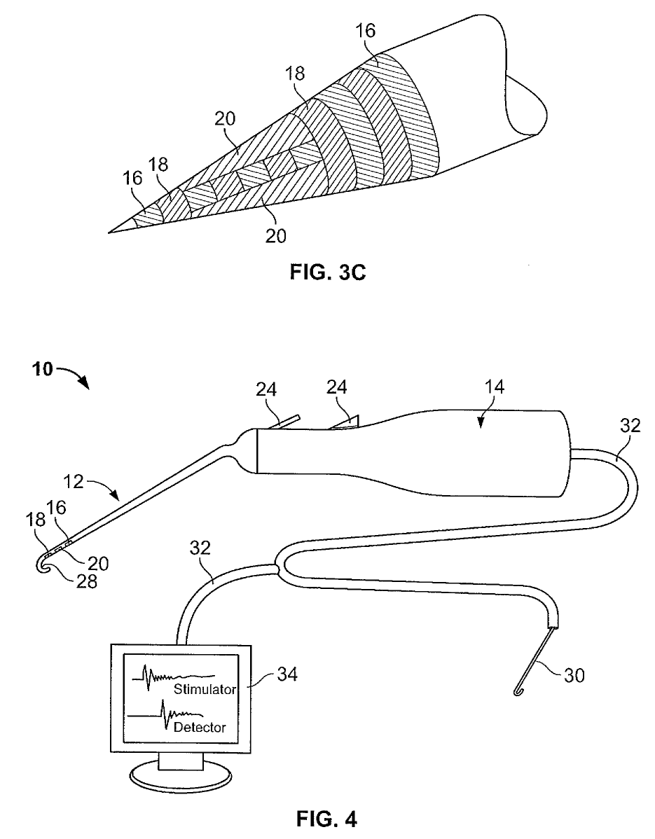

Referring now to FIG. 3A, 3B, and 3C that depict a close up partial view of some of the various engagement surfaces 28 of the probe assembly 12 of the surgical tool device 10. Also shown in FIG. 3A-3C are the low frequency LEDs 16, the high frequency LEDs 18, and a stimulator electrodes 20 attached to the probe assembly 12.

Referring now to FIG. 4 that depicts a perspective view of an assembled kit of the surgical tool device 10. The kit is shown to include the surgical tool device 10 having the probe assembly 12 coupled to a handle assembly 14. The probe assembly 12 is shown having a low frequency LED 16, a high frequency LED 18, and a stimulator electrode 20. The handle assembly 14 is shown attached to the probe assembly 12 and having a plurality of control switches 24. A cable 32 is shown attached to the handle assembly 14, to the monitoring system 34 and to the detector electrode probe 30.

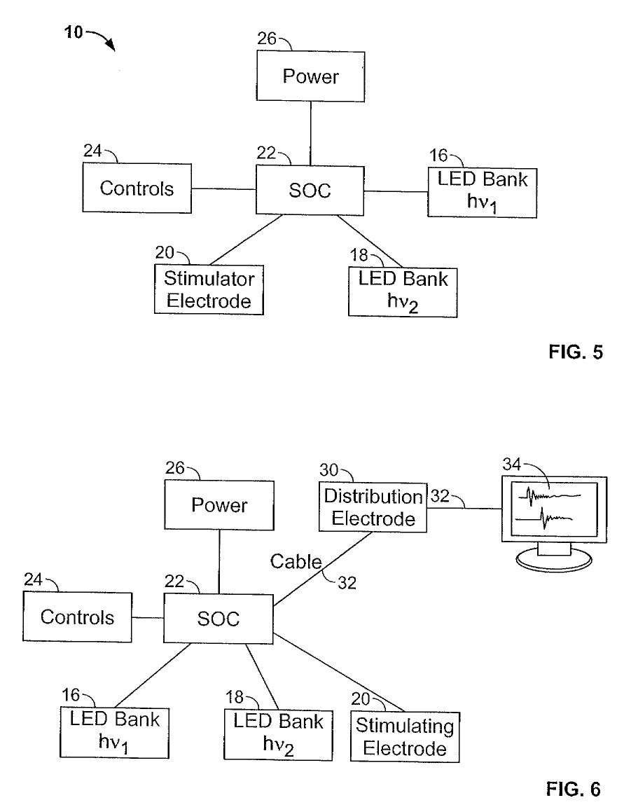

Referring now to FIG. 5 that depicts a logical communication scheme of the surgical tool device 10. The SOC 22 is shown to be in operative communication (i.e., electrically coupled to the power supply 26, to the low frequency LED 16, to the high frequency LED 18, to the stimulator electrode 20 and to the control switches 24.

Referring now to FIG. 6 that depicts a logical communication scheme of the kit for the surgical tool device. The SOC 22 is shown to be in operative communication (i.e., electrically coupled) to the power supply 26, to the low frequency LED 16, to the high frequency LED 18, to the stimulator electrode 20, to the control switches 24. The SOC 22 is also shown to be in operative communications, via the cable 32, with the detector electrode probe 30 and with the monitoring system 34.

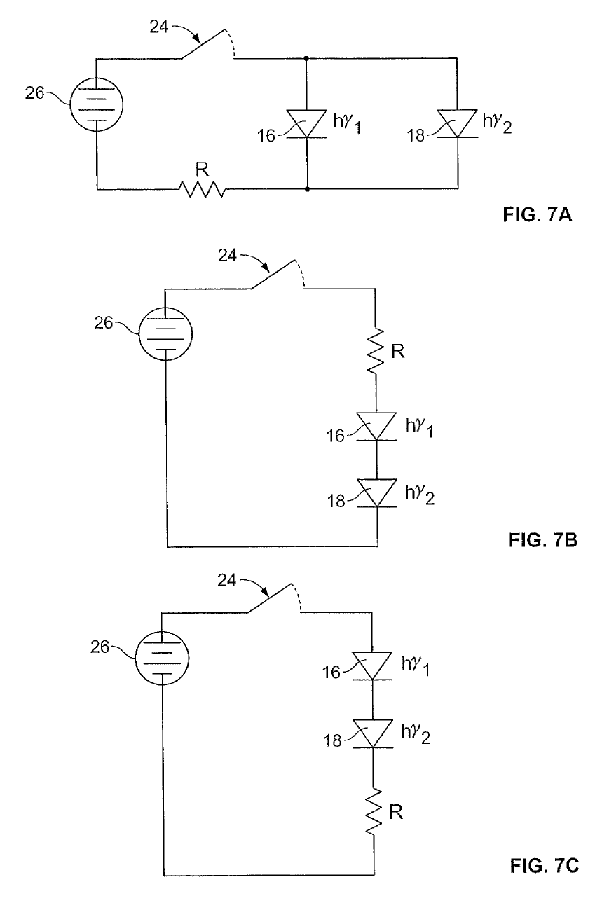

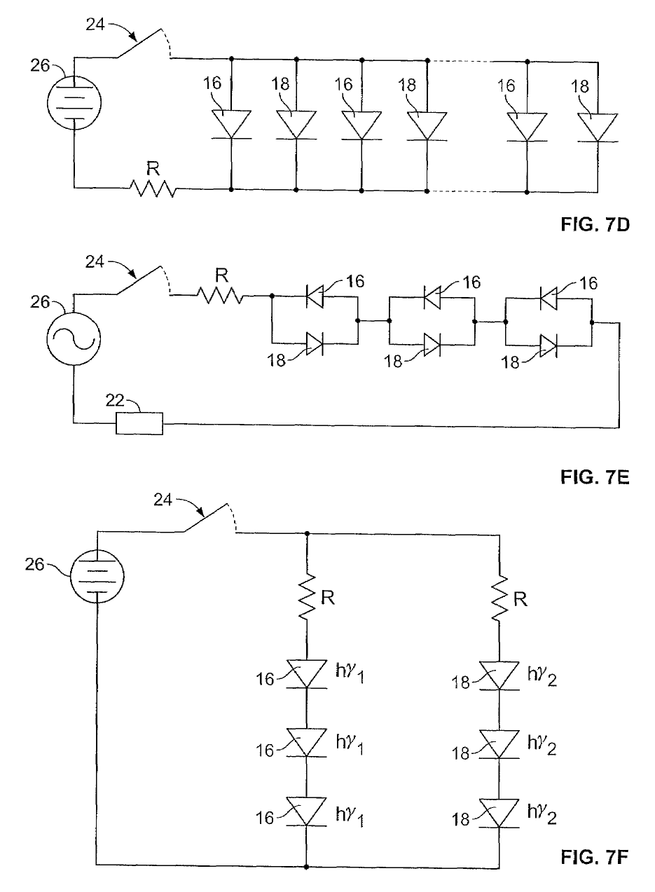

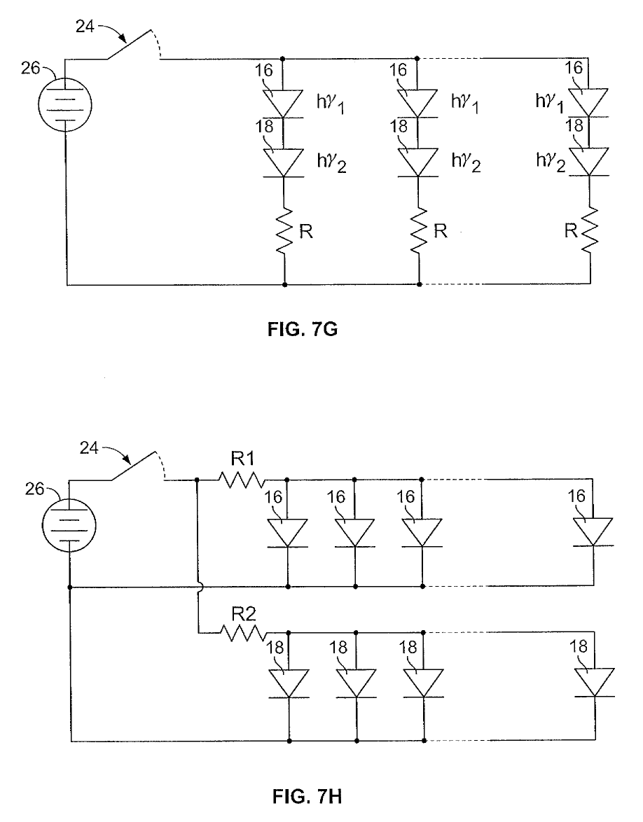

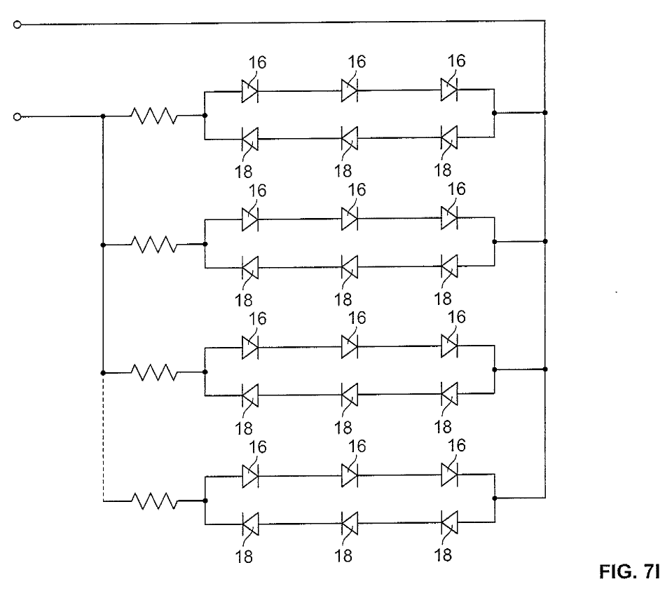

Referring now to FIGS. 7A, 7B, 7C, 7D, 7E, 7F, 7G, 7H, and 7I that depict various electronic schemes for driving the low and high frequency LEDS of the surgical tool device. The power supply 26 is shown to be in operative communication (i.e., electrically coupled) with the low frequency LEDs 16 and the high frequency LEDs 18.

As to the manner of usage and operation of the present invention, the same should be apparent from the above description. Accordingly, no further discussion relating to the manner of usage and operation will be provided.

While a preferred embodiment of the dual frequency LED/electrode surgical device has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or comprising or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A surgical tool device comprising:
a probe assembly having
a low frequency light emitting diode (LED) attached to the probe assembly;
a high frequency LED attached to the probe assembly; and
a stimulator electrode attached to the probe assembly; and
a handle assembly attached to the probe assembly, the handle assembly having:
a system on a chip (SOC) attached to the handle, the SOC electrically coupled to the low frequency LED, to the high frequency LED, and to the stimulator electrode.

2. The device of claim 1 further comprising a plurality of control switches attached to the handle, the control switches electrically coupled to the SOC, to the low frequency LED, to the high frequency LED, and to the stimulator electrode.

3. The device of claim 1 further comprising a power supply attached to the handle, the power supply electrically coupled to the SOC, to the low frequency LED, to the high frequency LED, and to the stimulator electrode.

4. The device of claim 1 wherein the low frequency LED is configured to emit light between red and infrared.

5. The device of claim 1 wherein the low frequency LED is configured to emit monochromatic light within a wavelength range between about 600 nm to about 1000 nm.

6. The device of claim 1 wherein the low frequency LED is configured to emit a spectral band of light of at least 10 nm wide within a wavelength range between about 600 nm to about 1000 nm.

7. The device of claim 1 wherein the low frequency LED is configured to emit light at about 1 μW/cm² to about 1 W/cm².

8. The device of claim 1 wherein the high frequency LED is configured to emit light between green and ultraviolet.

9. The device of claim 1 wherein the high frequency LED is configured to emit monochromatic light within a wavelength range between about 250 nm to about 550 nm.

10. The device of claim 1 wherein the high frequency LED is configured to emit a spectral band of light of at least 10 nm wide within a wavelength range between about 250 nm to about 550 nm.

11. The device of claim 1 wherein the high frequency LED is configured to emit light at about 1 μW/cm² to about 1 W/cm².

12. The device of claim 1 wherein the probe assembly having an arcuate engagement surface.

13. The device of claim 3 wherein the power supply is selected from the group consisting of a battery power supply and a high capacity capacitor power supply.

14. A surgical tool device comprising:
a probe assembly having
a low frequency light emitting diode (LED) attached to the probe assembly;
a high frequency LED attached to the probe assembly;
a stimulator electrode attached to the probe assembly; and
a system on a chip (SOC) attached to the probe assembly, the SOC electrically coupled to the low frequency LED, to the high frequency LED, and to the stimulator electrode; and
a handle assembly attached to the probe assembly.

15. A surgical tool kit comprising:
a probe assembly having:
a low frequency light emitting diode (LED) attached to the probe assembly;
a high frequency LED attached to the probe assembly; and
a stimulator electrode attached to the probe assembly;
a handle assembly configured to be attached to the probe assembly, the handle assembly having:
a system on a chip attached to the handle, the SOC configured to be electrically coupled to the low frequency LED, to the high frequency LED, and to the stimulator electrode;
a plurality of control switches attached to the handle, the control switches configured to be electrically coupled to the SOC, to the low frequency LED, to the high frequency LED, and to the stimulator electrode; and
a power supply attached to the handle, the power supply electrically coupled to the SOC, to the low frequency LED, to the high frequency LED, and to the stimulator electrode.

16. The kit of claim 15 further comprising a detector electrode probe configured to be electrically coupled to the SOC.

17. The kit of claim 16 further comprising a cable configured to electrically couple together the detector electrode probe to the SOC, and to the stimulator electrode.

18. The kit of claim 16 further comprising a monitoring system configured to be electrically coupled to the detector electrode.

19. A method of using a kit for a surgical tool device, the method comprising the steps of:
obtaining the kit comprising:
a probe assembly having:
a low frequency light emitting diode (LED) attached to the probe assembly;
a high frequency LED attached to the probe assembly; and
a stimulator electrode attached to the probe assembly;
a handle assembly configured to be attached to the probe assembly, the handle assembly having:
a system on a chip attached to the handle, the SOC configured to be electrically coupled to the low frequency LED, to the high frequency LED, and to the stimulator electrode;
a plurality of control switches attached to the handle, the control switches electrically coupled to the SOC; and
a power supply attached to the handle, the power supply electrically coupled to the SOC, and to the control switches;
a detector electrode probe configured to be electrically coupled to the SOC;
a cable configured to electrically couple together the detector electrode probe to the SOC, to the stimulator electrode; and
a monitoring system configured to be electrically coupled to the detector electrode;
attaching operatively together the probe assembly to the handle assembly so that the SOC is electrically coupled to the low frequency LED, to the high frequency LED, to the stimulator electrode, to the power supply, and to the control switches;
adjoining operatively together the cable to the handle and to the detector electrode so that the that the SOC is electrically coupled to the detector electrode; and
affixing together the detector electrode to the monitoring system so that the detector electrode and the stimulator electrode are operatively coupled to the monitoring system.

20. The method of claim 19 further comprising the steps of
making a opening into flesh near a nerve;
dispensing an aliquot of a photosensitizer into the opening;
inserting the probe assembly into an opening;
aligning the probe adjacent to the nerve while the probe is inserted into the opening;
pressing on one control switch to activate the stimulator electrodes to produce electrical impulses while the probe is aligned adjacent to the nerve;
stimulating the nerve with the electrical impulses;
displaying a response of the detector electrode while stimulating the nerve to identify the nerve and to verify the alignment of the probe;
irradiating low frequency light from the low frequency LED onto the nerve while the probe is aligned next to the nerve;
emitting high frequency light from the high frequency LED onto the nerve while the probe is aligned next to the nerve;
pulling the nerve aside with the probe assembly; and
withdrawing the probe assembly from the opening.
